(19) United States
(12) Patent Application Publication
(10) Pub. No.: US 2011/0201123 A1
(43) Pub. Date: Aug. 18, 2011

(54) LOW COST, HIGH ACCURACY OZONE SENSING

(75) Inventors: Eric K. Watson, Crestwood, KY (US); John Besore, Prospect, KY (US); Subhash Kollengode, Hyderabad (IN); Alexey Vert, Schenectady, NY (US); Sarah Kirchoff, Hartland, WI (US); Robert Strojan, Louisville, KY (US); Martin Austin, Portage, MI (US)

(73) Assignee: General Electric Company

(21) Appl. No.: 12/707,750

(22) Filed: Feb. 18, 2010

(51) Int. Cl. G01N 33/00; B01J 19/00

(52) U.S. Cl. 436/135; 422/129

(57) ABSTRACT

An ozone detection system includes a source of sample gas containing a concentration of ozone and a single optical pathway. The system includes a light source in optical communication with the optical pathway. The system further includes a first airflow passageway for receiving a first sample of gas from the source. The passageway includes a catalytic scrubber to reduce ozone content in the sample gas and passing the reduced ozone gas to a sensor. The system further includes a second airflow passageway for receiving a second sample of gas from the source and passing the gas unaltered to a sensor. The system further includes a sensor for sensing independently the light intensity of the sample of gas received from the first and second passageways. The system further includes a processor for receiving the light intensity data from the sensor and calculating the ozone concentration in the source of sample gas.

### BACKGROUND OF THE DISCLOSURE

The present disclosure relates to gas detection, more specifically to an ozone detection system and method. The ozone detection system finds particular application in consumer grade appliances, although it will be appreciated that selected aspects may find use in related applications encountering the same issues of a low cost ozone detection system capable of accuracy in the parts per billion range of ozone concentration.

Ozone (O₃) is a colorless gas which has both beneficial and detrimental effects on health and the environment. Ozone is a naturally occurring component resulting from a lighting storm giving that fresh air smell. It can be produced by the ultraviolet rays of the sun reacting with the Earth's upper atmosphere, which creates a protective ozone layer, or it can be created artificially with an ozone generator. However, ozone resulting from human activity at or close to the ground is a main component of smog, which adversely affects respiratory health, agricultural crops, and forests. While the ozone alone is not detrimental at ground level, it mixes with other more dangerous compounds. These other components of smog include nitrogen oxides (NOₓ), volatile organic compounds (VOC's), sulfur dioxide, acidic aerosols and gases, and particulate matter.

The ozone molecule contains three oxygen atoms whereas the oxygen molecule contains only two. Ozone is a very reactive and unstable gas with a short half-life before it reverts back to oxygen. Ozone is one of the most powerful and rapid acting oxidizers man can produce, and will oxidize most bacteria, mold and yeast spores, organic material and viruses.

Ozone is not only a very powerful oxidizing agent but also a very powerful non-chemical disinfectant. It has the unique feature of decomposing to a harmless nontoxic environmentally safe material, namely oxygen. In Europe, ozone is used for many purposes: color removal, taste and odor removal, turbidity reduction, organics removal, micro flocculation, iron and manganese oxidation, and most commonly, bacterial disinfection and viral inactivation. Most of these applications are based on ozone's high oxidizing power. When used in water treatment, ozone can be introduced at different points in the process, depending on its intended application. For iron and manganese oxidation or to induce flocculation, it is usually introduced early, and when used for taste and odor removal it is introduced at an intermediate point. In European water treatment practices, ozonation is recognized as a preferred method of virus inactivation rather than just an alternative to the use of chlorine for disinfection.

Nine out of ten diseases, including the common cold and the flu, are caused by water or airborne bacteria and viruses. Like chlorine, ozone kills microorganisms. The sterilization action of ozone is by “direct kill attack” and oxidation of the biological material. The rate of bacteria killed by ozone is 3500 times faster than with chlorine. Virus destruction with ozone is instantaneous, safe and foolproof, as ozone is nature's own purifier. Chlorine's reactive oxidant is hypochloric acid which is formed when chlorine is dissolved in water. This powerful oxidant will have significant long term negative effects on our water sources. Ozone, on the other hand, has no side effects as far as the treatment of water is concerned. It has properly been described as the “add-nothing” sterilant.

Current ozone sensing technologies are not easily adapted to work in consumer grade appliances. Some technologies are cross-sensitive. While others such as electrochemical type sensors are very expensive and last only approximately two years. UV Photometry is not cross-sensitive and will last as long as the UV-C band source and detector work. However, existing UV Photometers are expensive. Thus, there exists a need to overcome these problems in order to provide for an ozone detection system useful in consumer grade appliances.

Even in light of recent advances, the industry continues to lack a low cost ozone system useful in consumer grade appliances and capable of accuracy in the parts per billion range of ozone concentration.

### SUMMARY OF THE DISCLOSURE

In one aspect, the present disclosure relates to an ozone detection system for detecting the ozone concentration in a sample gas, e.g., ozone laden air, which includes a source of sample gas containing a concentration of ozone. The system further includes a single optical pathway and a light source in optical communication with the optical pathway. The system further includes a first airflow passageway for receiving a first sample of gas from the source. The passageway includes a catalytic scrubber to reduce ozone content in the sample gas and passing the reduced ozone gas to a sensor. The system further includes a second airflow passageway for receiving a second sample of gas from the source and passing the gas unaltered to a sensor. The system further includes a sensor for sensing independently the light intensity of the sample of gas received in the first and second passageways. The system further includes a processor for receiving the light intensity data from the sensor and calculating the ozone concentration in the source of sample gas.

In another aspect, the light source is a UV-C band LED.

In another aspect, the optical pathway has a length of about 13 inches to about 18 inches.

In another aspect, the catalytic scrubber includes the catalyst manganese oxide.

In yet another aspect, the sensor is a photo-detector wherein the photo-detector is a silicon semi-conductor photodiode which includes a bandpass filter such as a quartz window.

In yet another aspect, the present disclosure relates to a method of forming an ozone detection system for use in a consumer grade appliance. The method involves providing a source of sample gas containing a concentration of ozone and a single optical pathway. The method further includes providing a light source in optical communication with the optical pathway. The method further includes providing a first airflow passageway for receiving a first sample of gas from the source. The passageway includes the catalytic scrubber to reduce ozone content and the sample gas and passing the reduced ozone gas to a sensor. The method further includes providing the second airflow passageway for receiving a second sample of gas from the source and passing the gas unaltered to a sensor. The method further provides for a sensor for sensing independently the light intensity of the sample of gas received from the first and second passageways. The method further includes providing a processor for receiving the light intensity data from the sensor and calculating the ozone concentration in the source of sample gas.

In yet another aspect, the present disclosure relates to an ozone detection system for detecting the ozone concentration in a sample gas, e.g., ozone laden air, which includes a source of sample gas containing a concentration of ozone and a single optical pathway. The system further includes a light source in optical communication with the optical pathway, wherein the light source is a UV-C band LED. The system further includes a first airflow passageway for receiving a first sample of gas from the source. The passageway includes a catalytic scrubber to reduce ozone content in the sample gas and passing the reduced ozone gas to a sensor. The system further includes a second airflow passageway for receiving a second sample of gas from the source and passing the gas unaltered to a sensor. The system further includes a sensor for sensing independently the light intensity of the sample of gas received from the first and second passageways. The system further includes a processor for receiving the light intensity data from the sensor and calculating the ozone concentration in the source of sample gas.

In yet another aspect, the present disclosure relates to an ozone detection system for determining the ozone concentration in a sample gas, e.g., ozone laden air, which includes a single optical pathway. The system further includes a light source in optical communication with the optical pathway. The system further includes a sensor to sense the light intensity in the pathway. The system further includes first and second sample inlet ports in communication with the optical pathway for receiving the sample gas which diverge into a first passageway and a second passageway. The first passageway includes a catalytic scrubber to reduce the ozone concentration of the sample gas flowing therethrough to deliver a reference gas to the optical pathway. The second passageway delivers the sample gas to the optical pathway. The system further includes a valve operative to connect the first and second passageways to the optical pathway respectively. The system further includes a processor operative to control the state of the valve which receives the light intensity data from the sensor representing light intensity when the reference gas is in the pathway and when the sample gas is in the pathway. The system is further operative to calculate an actual ozone concentration. The sample gas is a function of this data

A primary benefit realized by the ozone detection system is a low cost system for use in a consumer grade appliance and capable of accuracy in the parts per billion range of ozone concentration.

Another benefit realized by the ozone detection system is the ability to use a single pathway for taking a reference reading at the beginning of the sample period of a reduced gaseous ozone sample, and using this reference reading throughout the measuring period.

Another benefit realized by the ozone detection system is the use of a narrow band UV LED.

Still other features and benefits of the ozone detection system according to the invention will become more apparent from reading and understanding the following detailed description.

### BRIEF DESCRIPTION OF THE DRAWINGS

FIG. 1 is a flow diagram of an ozone detection system according to an exemplary embodiment; and

FIG. 2 is a diagram of an ozone detection system with a cross-sectional view of an exemplary single optical pathway according to an exemplary embodiment.

### DETAILED DESCRIPTION OF THE PREFERRED EMBODIMENTS

Unexpectedly, the current inventive ozone detection system is a low cost, solid state, tubing and electronic system with the ability to measure ozone concentrations with accuracy in the parts per billion. The system can be at least about 50 percent more cost effective, e.g. 80 percent cost effective compared to current technology on the market. Therefore, this allows for the control of ozone concentration during deodorizing and sanitization cycles in a consumer grade appliance. For example, the current ozone sensing system may be incorporated into a deodorizing- or sanitizing-type consumer product such as those products set forth in U.S. Ser. No. 12/621,947 to our common assignee, among others. The ability of the system to measure ozone concentration results from a combination of ultraviolet UV photometry using UV-C band solid state light emitting diodes (LEDs) and computing the concentration of ozone with the Beer-Lambert Law. As used here, the term “tubing” refers to any closed pathway through which the target sample ozone-containing gas may pass as part of the sensing process.

Gases in general have at least one defined peak of absorption at a particular wavelength corresponding to a particular component of the gas. For ozone-containing gases or samples, the one peak of absorption for ozone is at 254 nm due to an internal electronic resonance of the ozone molecule. The invention utilizes UV photometry techniques to determine the specific absorption cross-section of the ozone in the sample, and then uses the Beer-Lambert law to determine the concentration. In use, the ozone detector disclosed herein, in the initial cycle, passes a sample of the air to be tested through a scrubber having manganese dioxide disposed therein to remove ozone from the sample. The scrubbed sample air then enters the sample absorption cell to establish a reference light intensity at zero ozone concentration (Iₒ). In the second part of the cycle, sample air is directed to bypass the scrubber and enters the sample cell directly for measurement of the attenuated light intensity (I). The difference between the light intensity of the scrubbed, ozone reduced gas and the light intensity of the unscrubbed sample is related(?) to the ozone concentration according to the Beer-Lambert law.

The Beer-Lambert Law is represented by the following equation:

$$C = ( \frac{1}{KL} ) \times ( \frac{{P}_{STD}}{{P}_{BARO}} ) \times ( \frac{{T}_{C} + 273.15}{{T}_{STD}} ) \times ln ( \frac{{I}_{O}}{I} )$$

wherein:

| K = | 308.32 1/cm 1/ATM | | Molecular absorption coefficient |
|---|---|---|---|
| L = | 40.64 | cm | Path length of cell (cm) |
| P_{STD} = | 760 | mmHg | Standard pressure at 1 ATM |
| P_{BARO} = | 760 | mmHg | Sample Pressure (not used in this |
| | | | implementation) |
| T_{C} = | 25 | C. | Temperature of sample |
| T_{STD} = | 273.15 | K | Standard temperature at 1 ATM |
| I_{O} = | | | UV Light intensity of sample without |
| | | | Ozone (reference gas) |
| I = | | | UV Light intensity of sample with |
| | | | Ozone (sample gas) |

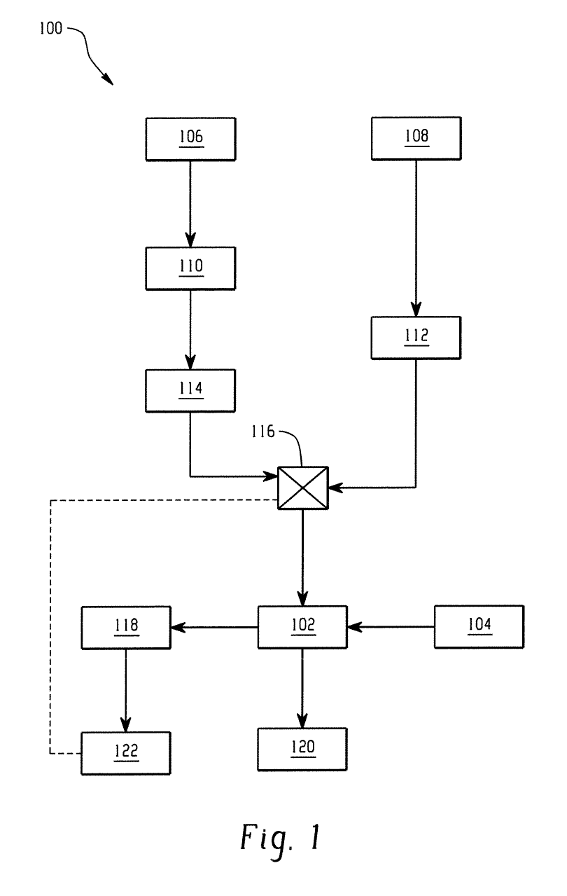

FIG. 1 is a flow diagram of an exemplary ozone detection system 100 in accord with the invention. The ozone detection system 100 includes a single optical pathway 102, a light intensity sensor 118 and a light source 104 in optical communication with sensor 118 through the pathway 102. In this embodiment, the light source is a UV-C band LED. Although, it may be appreciated the light source may be of another type configured to a particular consumer grade appliance.

The system includes first and second sample inlet ports 106, 108 which diverge into first and second airflow passageways 110, 112. In an exemplary embodiment, there are two sample inlet ports. In other embodiments, there may be one sample inlet port in communication with first and second airflow passageways 110, 112 respectively. The first airflow passageway 110 includes a catalytic scrubber 114 to reduce the ozone. The catalytic scrubber 114 contains at least a catalytic material which is used to remove ozone in order to establish a reference reading to compute the ozone concentration using the Beer-Lambert Law. The catalytic material may be a metal oxide catalyst selected from the group of manganese dioxide, cobalt oxide, copper oxide, nickel oxide, and combinations thereof. In an exemplary embodiment, the catalytic material is the metal oxide catalyst manganese dioxide. It is known in the art that a metal oxide catalyst, such as manganese dioxide, may be used for this purpose and that it typically reduces the ozone level by at least about 50 percent, and generally less than about 95 percent, e.g. about 65 percent.

Ozone-laden air is drawn from the first and second airflow passageways 110, 112 through a valve 116. Valve 116 is operative in a first state to direct the gaseous air into the airflow passageway 108 and in a second state to direct the ozone-laden air into passageway 110. For purposes of this application the word valve refers to any type of device known to shut off, release, dose, distribute or mix fluids or gases. For example, one such valve may be a solenoid valve. The ozone-containing sample directed into passageway 110 passes through the scrubber 114 to the reduce ozone content in the sample, producing a substantially ozone free reference gas. The scrubbed reference gas is drawn using an air pump 120 through the solenoid valve 116 into the optical pathway 102 to establish the reference light intensity at zero ozone concentration I₀. The degree of reduction in ozone content is based on the capacity of the catalytic scrubber to separate (reduce) the O₃ into O₂.

The path length L of the optical pathway 102 determines the resolution to which the instrument can determine the level of ozone in the sample. As the path length L increases, the resolution becomes more precise. However, the source light is the limiting factor on the maximum path length. The length L of the pathway 102 for an exemplary embodiment, for example, can be at least about 13 inches, generally less than about 22 inches, e.g., about 16 inches. Once the reference light intensity I₀ has been established, gaseous ozone air is drawn into sample inlet port 108, bypassing passage 110 and scrubber 114, into the airflow passageway 112. The air pump 120 draws the gaseous ozone air into the optical pathway 102 through solenoid valve 116 for measurement of the light intensity I which is attenuated by the presence of ozone. The sensor 118 is positioned with respect to the light source 104 such that it can measure the intensity of the light passing through pathway 102.

The sensor 118, therefore, when the valve directs air from passageway 112 to the optical pathway 102, measures the light intensity at 254 nm of the ozone laden sample gas from the passageway 112, which is attenuated as compared to the reference light intensity at zero ozone concentration I₀ due to the presence of the ozone. The sensor 118 may be a photodetector in the form of a semi-conductor photodiode. It may be appreciated that other sensors in the form of photo-detectors may be used. The photo-detectors may be selected from the group consisting of photodiodes, phototransitors, photodarlingtons, photomultiplier tubes, photoresistors, integrated circuits, hybrids thereof, and combinations thereof. In an exemplary embodiment, the semi-conductor photodiode may include a quartz window or any other material suitable for use as a bandpass filter at the appropriate wavelength. For wavelengths below 300 nm, photodiodes with quartz windows are preferably used given that borosilicate glass or a plastic resin coated glass tends to block the shorter wavelength light. Therefore, in one embodiment, the photodiode predominantly comprises a non-metal selected from the group of silicon, germanium, gallium, arsenide, indium, antimonite, phosphorus, and combinations thereof. In an exemplary embodiment, the photodiode comprises silicon.

The photodetector 118, subsequent to measuring light intensity, transfers the data to processor 122 which receives the data and calculates the actual ozone concentration using, as described above, the Beer-Lambert Law.

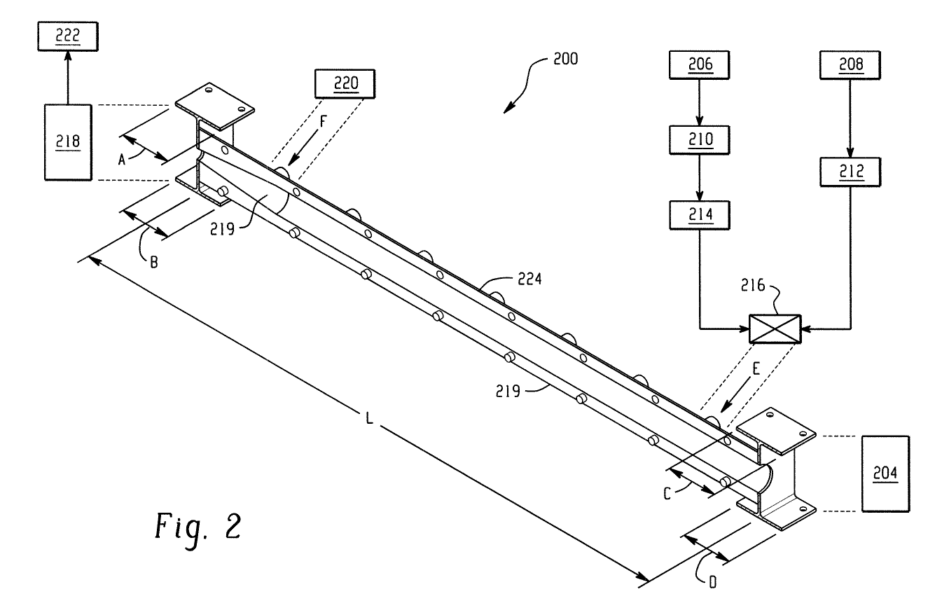

In accord with one embodiment of the invention, FIG. 2 illustrates a cross-sectional view of an exemplary ozone detections system 200. The ozone detection system 200 includes a single optical pathway 202 and a light source 204 in communication with the pathway 202. Two structures fitted together at edges A, B, C, and D faun the complete optical pathway. In an exemplary embodiment, first and second sample inlet ports 206, 208 diverge into first and second airflow passageways 210, 212. The length L of the pathway 202 for an exemplary embodiment, for example, can be at least about 13 inches, generally less than about 22 inches, e.g., about 16 inches. The UV-C band LED light source 204 is located in communication with the optical pathway 202. As described above, a valve 216 may be used to direct the gaseous air flow (or any valve or type of device known to shut off, release, dose, distribute or mix fluids or gases) either through path 210 or 212. An air pump 220 draws air from the first and second airflow passageways through the valve 216 into the optical pathway 202. Closed holes E, F can be opened along the optical pathway 202 connecting the air pump 220 and valve 216 to the optical pathway 202 using tubing. At the other end of the pathway, the detector 218 is located proximate to optical pathway 202 comprising a narrowed diameter portion 219. The narrowed diameter portion 209 is used to focus all light energy into the diameter of the photodetector cell. The optical pathway 202 can be formed from a composition selected from the group of plastic extrusions, such as, clear polyvinyl chloride, polycarbonate, polystyrene, polysulfone, other custom extruded plastics and combinations thereof. In an exemplary embodiment, the optical pathway 202 is formed from polycarbonate which is then coated with aluminum and top-coated with silicon dioxide. The gloss level for the outer surface 221 of the optical pathway 202 for an exemplary embodiment, for example, can be at least about 85 degrees, generally less than about 95 degrees, e.g., about 90 degrees. Gloss level or reflectivity in substantially this bandwidth of 254 nm, otherwise the light energy may not be able to travel down the tube efficiently.

The photodetector 218, subsequent to measuring the light intensity, transfers the data to processor 222 which receives the data and calculates the actual ozone concentration using, as described above, the Beer-Lambert Law.

The invention has been described with reference to the preferred embodiments. Obviously, modifications and alterations will occur to others upon reading and understanding the preceding detailed description. It is intended that the invention be construed as including all such modifications and alterations.

Having thus described the invention, it is now claimed:

1. An ozone detection system comprising:
a source of sample gas containing a concentration of ozone;
a single optical pathway;
a light source in optical communication with the optical pathway;
a first airflow passageway for receiving a first sample of gas from the source, the passageway comprising a catalytic scrubber to reduce ozone content in the sample gas and passing the reduced ozone gas to a sensor;
a second airflow passageway for receiving a second sample of gas from the source and passing the gas unaltered to a sensor;
a sensor for sensing independently the light intensity of the sample of gas received from the first and second passageways; and
a processor for receiving the light intensity data from the sensor and calculating the ozone concentration in the source of sample gas.

2. The system of claim 1 wherein the light source is a UV-C band LED.

3. The system of claim 1 wherein the optical pathway has a length of about 13 inches to about 22 inches.

4. The system of claim 1 wherein the optical pathway has a length of about 16 inches.

5. The system of claim 1 wherein the catalytic scrubber contains a metal oxide catalyst selected from the group consisting of manganese oxide, cobalt oxide, copper oxide, nickel oxide, and combinations thereof.

6. The system of claim 5 wherein the catalyst is manganese oxide.

7. The system of claim 1 wherein the sensor is a photo-detector.

8. The system of claim 7, wherein the photo-detector is a semi-conductor photodiode comprising a bandpass filter.

9. The system of claim 8 wherein the semi-conductor photodiode comprises a non-metal selected from the group consisting of silicon, germanium, gallium, arsenide, indium, antimonite, phosphorus, silicon carbide, and combinations thereof.

10. The system of claim 8 wherein the semi-conductor photodiode is silicon.

11. A method to detect ozone concentration for use in a consumer grade appliance providing ozone-containing gas comprising:
providing a source of sample gas containing a concentration of ozone;
providing a single optical pathway;
providing a light source in optical communication with the optical pathway;
providing a first airflow passageway for receiving a first sample of gas from the source, the passageway comprising a catalytic scrubber to reduce ozone content in the sample gas and passing the reduced ozone gas to a sensor;
providing a second airflow passageway for receiving a second sample of gas from the source and passing the gas unaltered to a sensor;
providing a sensor for sensing independently the light intensity of the sample of gas received from the first and second passageways; and
providing a processor for receiving the light intensity data from the sensor and calculating the ozone concentration in the source of sample gas.

12. The method of claim 11, providing wherein the light source is a UV-C band LED.

13. The method of claim 11, providing the optical pathway includes having a length of about 13 inches to about 22 inches.

14. The method of claim 11 providing the optical pathway has a length of about 16 inches.

15. The method of claim 11, providing the catalytic scrubber contains a metal oxide catalyst selected from the group consisting of manganese oxide, cobalt oxide, copper oxide, nickel oxide, and combinations thereof.

16. The method of claim 15 providing the catalyst is manganese oxide.

17. The method of claim 11 providing the sensor is a photo-detector.

18. The method of claim 17 providing the photo-detector is a semi-conductor photodiode comprising a bandpass filter.

19. The method of claim 18 providing the semi-conductor photodiode comprises a non-metal selected from the group consisting of silicon, germanium, gallium, arsenide, indium, antimonite, phosphorus, silicon carbide, and combinations thereof.

20. The method of claim 18 providing the semi-conductor photodiode is silicon.

21. An ozone detection system comprising:
a source of sample gas containing a concentration of ozone;
a single optical pathway;
a light source in optical communication with the optical pathway, wherein the light source is a UV-C band LED;
a first airflow passageway for receiving a first sample of gas from the source, the passageway comprising a catalytic scrubber to reduce ozone content in the sample gas and passing the reduced ozone gas to a sensor;
a second airflow passageway for receiving a second sample of gas from the source and passing the gas unaltered to a sensor;
a sensor for sensing independently the light intensity of the sample of gas received from the first and second passageways; and
a processor for receiving the light intensity data from the sensor and calculating the ozone concentration in the source of sample gas.

22. The system of claim 21 wherein the optical pathway includes having a length of about 13 inches to about 22 inches.

23. The system of claim 21 wherein the sensor is a photo-detector.

24. The system of claim 23, wherein the photo-detector is a silicon semi-conductor photodiode comprising a bandpass filter.

25. An ozone detection system for determining the ozone concentration of a sample gas comprising:
a single optical pathway;
a light source in optical communication with the optical pathway;
a sensor to sense the light intensity in the pathway;
first and second sample inlet ports in communication with the optical pathway for receiving the sample gas, which diverge into a first passageway and second passageway, the first passageway comprising a catalytic scrubber to reduce the ozone concentration of the sample gas flowing therethrough to deliver a reference gas to the optical pathway; the second passageway delivers the sample gas to the optical pathway;
a valve operative to connect the first and second passageways to the optical pathway respectively; and
a processor operative to control the state of the valve which receives the light intensity data from the sensor representing light intensity when the reference gas is in the pathway and when the sample gas is in the pathway and is further operative to calculate an actual ozone concentration of the sample gas as a function of this data.

26. The system of claim 25 wherein the light source is a UV-C band LED.

27. The system of claim 25 wherein the optical pathway has a length of about 13 inches to about 22 inches.

28. The system of claim 25 wherein the optical pathway has a length of about 16 inches.

29. The system of claim 25, wherein the catalytic scrubber contains a metal oxide catalyst selected from the group consisting of manganese oxide, cobalt oxide, copper oxide, nickel oxide, and combinations thereof.

30. The system of claim 29 wherein the catalyst is manganese oxide.

31. The system of claim 24 wherein the sensor is a photo-detector.

32. The system of claim 31 wherein the photo-detector is a semi-conductor photodiode comprising a bandpass filter.

33. The system of claim 32 wherein the semi-conductor photodiode comprises a non-metal selected from the group consisting of silicon, germanium, gallium, arsenide, indium, antimonite, phosphorus, silicon carbide, and combinations thereof.

34. The system of claim 32 wherein the semi-conductor photodiode is silicon.
